(19) United States
(12) Patent Application Publication
(10) Pub. No.: US 2019/0175202 A1
(43) Pub. Date: Jun. 13, 2019

(54) ELECTRONIC ASPIRATOR

(71) Applicant: Rudolph Piatyszek, Miramar, FL (US)

(72) Inventor: Rudolph Piatyszek, Miramar, FL (US)

(21) Appl. No.: 16/321,978

(22) PCT Filed: Jul. 27, 2017
(86) PCT No.: PCT/US17/44084

§ 371 Date: Jan. 30, 2019

Related U.S. Application Data
(60) Provisional application No. 62/369,078, filed on Jul. 30, 2016.

(51) Int. Cl. A61B 17/24; A61M 1/00

(52) U.S. Cl. CPC A61B 17/24; A61M 1/0003; A61M 1/0049; A61M 2210/0618; A61B 2217/005; A61M 2205/07; A61B 2017/246

(57) ABSTRACT

An aspirator including an impeller driven by a driving component to create a suction through a fluid inlet; a housing surrounding the impeller; and a disposable tip. The disposable tip is removeably couplable to the housing at the fluid inlet, defines a fluid intake port in fluid communication with the outside environment and the fluid inlet, and has an aspirated bodily fluid collection region that collects and retains aspirated bodily fluids drawn into the disposable tip by the impeller's suction.

### CROSS-REFERENCE TO RELATED APPLICATION

This application claims priority to U.S. Provisional Patent Application No. 62/369,078 filed Jul. 30, 2016, the entirety of which is incorporated by reference, and to application no. PCT/US17/44084 filed Jul. 27, 2017 under the Patent Cooperation Treaty, the entirety of which is also incorporated by reference.

### FIELD OF THE INVENTION

The present invention relates generally to aspirators, and, more particularly, relates to electronic aspirators for removing bodily fluids and other bodily wastes from patients.

### BACKGROUND OF THE INVENTION

Nasal congestion is a common problem newborns and young children have causing obstruction of their upper airways and preventing them from breathing normally Nasal congestion, if left untreated, can affect their sleep, hearing, and speech development, and can even cause problems such as respiratory distress and sleep apnea. Due to the child's inability to remove the mucous themselves, aspirators are used to assist the child.

One of the oldest and most common types of mechanical aspirators is a bulb syringe. These bulb syringes require manual pumping and are typically small and limited in the amount of bodily fluids they can suck/draw in per manual pumping action. Accordingly, multiple pumps are generally needed to remove the obstruction. In addition, due to their small size, the suction created by such bulb syringes is also much weaker than other aspirators, creating issues in completely clearing the child's nostril of mucous.

Along with the different variations of bulb syringes in existence, another common mechanical aspirator requires an operator to use his/her own suction to draw out mucous from the child's nose. Stated another way, the operator in such mechanical aspirators physically suck in mucous with his/her mouth through a long tube, from the child's nostrils. A filter is placed within the tube to prevent the operator from sucking the child's mucous into the operator's mouth. Such aspirators are generally considered more powerful than the bulb syringe because they utilize the much larger and more powerful human lungs to create suction. With that being said, some of the drawbacks of such devices are that they require replacement filters and present a potential hazard in contracting viral and/or bacterial infections if the filter is not functioning properly and/or if the operator did not install the filter properly. In addition, many operators (e.g., parents) find such a method of removing mucous using the operator's own mouth to be undesirable and distasteful.

Yet other types of known aspirators are electronic aspirators that utilize a pump system in which a diaphragm is pushed in and out to suck in mucous. The diaphragm used in these pump system aspirators are significantly smaller than the bulb syringes and simply do not create a strong enough suction force to suck in bodily fluids. It is generally effective only if there is a complete seal between the tip of the device and the nostril and/or if the tip is fully submersed in the bodily fluid/waste.

One drawback that all these known devices have in common is that they all require cleaning after use, which, if not properly cleaned, can cause the devices to become contaminated, and/or can become unusable as the waste storing chambers will eventually become full.

Therefore, a need exists to overcome the problems with the prior art as discussed above.

### SUMMARY OF THE INVENTION

The invention provides an electronic aspirator that overcomes the hereinbefore-mentioned disadvantages of the heretofore-known devices and methods of this general type.

With the foregoing and other objects in view, there is provided, in accordance with the invention, an aspirator including a housing having a suction inlet; an impeller disposed within the housing, the impeller being driven by a driving component to create a suction through the suction inlet; and a disposable tip. The disposable tip may be removeably couplable to the housing at the suction inlet, define a fluid intake port in fluid communication with an outside environment and the suction inlet, and may have an aspirated bodily fluid collection region that collects aspirated bodily fluids drawn into the disposable tip by the impeller's suction and retains the aspirated bodily fluids by changing a direction of a flow of the aspirated bodily fluids within the aspirated bodily fluid collection region.

In accordance with another embodiment of the present invention, at least a portion of the disposable tip is formed as a nasal insertion tip, the nasal insertion tip being dimensioned and configured for insertion with a nostril.

In accordance with yet another embodiment of the present invention, the aspirated bodily fluid collection region includes barriers disposed to direct a flow of the aspirated bodily fluids to reverse direction within the disposable tip.

In accordance with another embodiment of the present invention, the aspirated bodily fluid collection region includes barriers disposed to direct a flow of the aspirated bodily fluids in a zig-zag pattern within the disposable tip.

In accordance with yet another embodiment of the present invention, the aspirated bodily fluid collection region includes a multidirectional chamber defining a multidirectional bodily fluid passageway within the disposable tip, the multidirectional bodily fluid passageway having at least two separate portions along a length of the passageway that at least partially overlap one another within the disposable tip.

In accordance with yet another feature, an embodiment of the present invention further includes a plurality of blades and is fixedly coupled to a rotation shaft of the driving component.

In accordance with another feature of the present invention, the impeller defines a center inlet and includes a plurality of blades disposed around the center inlet, the plurality of blades being operably configured to push air in a radially outward direction during a rotary motion of the impeller.

In accordance with yet another embodiment of the present invention, the housing at least partially defines an airflow discharge passage fluidly coupling the impeller to at least one exhaust port disposed at a distal end of the housing opposite a proximal end of the housing at which the impeller is disposed; and the impeller includes a plurality of blades operable to direct airflow created by the impeller through the airflow discharge passage and to the outside environment via the at least one exhaust port.

In accordance with yet another embodiment of the present invention, the driving component is formed as a motor; and the housing includes at least one exhaust port at a distal end of the housing, the at least one exhaust port disposed a sufficient distance from the impeller and the motor such that noise from the impeller and the motor is below 85 decibels.

In accordance with yet another feature, an embodiment of the present invention further includes a vibration-dampening material disposed around the driving component and configured to absorb vibrations created by the driving component.

In accordance with yet another feature, an embodiment of the present invention further includes a decorative body removeably couplable to the housing at the suction inlet and defining an opening for receiving at least a portion of the disposable tip through the opening into the outside environment.

In accordance with yet another feature of the present invention, the decorative body includes an animal face surface disposed to face away from the housing such that the animal face surface is viewable by a patient in a nasal aspiration use-configuration.

In accordance with another feature of the present invention, the housing includes an exterior surface, at least a portion of the exterior surface formed as a handle portion with a user gripping surface disposed to receive an operator's hand grip to guide and hold at least a portion of the disposal tip into a patient's nostril.

In accordance with yet another feature, an embodiment of the present invention further includes an actuator button disposed on the housing and operable to selectively rotate the impeller to create the suction.

In accordance with another feature, an embodiment of the present invention includes an apparatus with a disposable nasal tip, the disposable nasal tip being removeably couplable to an aspirator at a suction inlet of the aspirator; defining a fluid intake port in fluid communication with an outside environment and the suction inlet; and having an aspirated bodily fluid collection region with non-filter barriers disposed to collect and retain aspirated bodily fluids drawn into the disposable nasal tip by a suction force from the aspirator, the non-filter barriers retaining the aspirated bodily fluids by changing a direction of a flow of the aspirated bodily fluids within the disposable nasal tip.

In accordance with yet another feature of the present invention, the non-filter barriers are disposed to direct a flow of the aspirated bodily fluids to reverse direction within the disposable tip.

In accordance with a further feature of the present invention, the non-filter barriers are disposed to direct a flow of the aspirated bodily fluids in a zig-zag pattern within the disposable tip.

In accordance with yet another feature, an embodiment of the present invention includes an aspirator kit with an aspirator device and at least one disposable tip. The aspirate device may include a suction inlet, an impeller driven by a driving component to create a suction through the suction inlet, and a housing surrounding the impeller and the driving component. The disposable tip may be removeably couplable to the housing at the suction inlet, define a fluid intake port in fluid communication with an outside environment and the suction inlet, and have an aspirated bodily fluid collection region that collects and retains aspirated bodily fluids drawn into the disposable tip by the impeller's suction by changing a direction of a flow of the aspirated bodily fluids within the disposable tip.

In accordance with a further feature of the present invention, the aspirator kit may further include at least one decorative body, each of the decorative bodies being removeably couplable to the aspirator device and defining an opening for receiving at least a portion of the disposable tip through the opening into the outside environment. In yet a further feature of the present invention, each of the decorative bodies includes an animal face surface facing away from the housing and each of the least one decorative body having a different animal face surface from the other ones of the decorative bodies.

Although the invention is illustrated and described herein as embodied in an electronic aspirator, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the housing. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a cross-sectional side view of an electronic aspirator in accordance with the present invention;

FIG. 2 is a cross-sectional side view of a disposable tip removeably couplable to the electronic aspirator of FIG. 1, in accordance with the present invention;

FIG. 3 is a front view of an assembled aspirator assembly with the disposable tip of FIG. 2 coupled to the electronic aspirator of FIG. 1 and a decorative head portion, in accordance with an embodiment of the present invention;

FIG. 4 is a perspective side view of the assembled aspirator assembly shown in FIG. 3, in accordance with the present invention;

FIG. 5 is a perspective rear view of the assembled aspirator assembly shown in FIG. 3 in use with a child, in accordance with an embodiment of the present invention;

FIG. 6 is a block diagram of at least a portion of the electronic components within the electronic aspirator of FIG. 1, in accordance with an exemplary embodiment of the present invention;

FIG. 7 is a flow chart depicting an exemplary method of using the aspirator of FIG. 1 to remove mucous from a nasal cavity, in accordance with an embodiment of the present invention; and

FIG. 8 is a downward-looking perspective view of an alternative impeller that may be used in the electronic aspirator of FIG. 1, in accordance with one embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient electronic aspirator. Embodiments of the invention provide an electronic portable aspirator that includes an impeller, which provides a suction force that is stronger than conventional bulb syringes, diagram-pump aspirators, and other known aspirators. In addition, embodiments of the invention provide an aspirator with a disposable tip removeably couplable to the housing of the aspirator and designed to trap bodily fluids, such as mucous, within the disposable tip for easy clean-up and so that such bodily fluids do not enter the aspirator housing. Additional embodiments of the invention provide for a removable and interchangeable decorative face that can comfort patients (e.g., children) and/or an audio system that can selectively upload and play music files to further comfort/distract the patient from the bodily fluid removal process. Yet other embodiments of the invention provide for noise reduction and vibration dampening features.

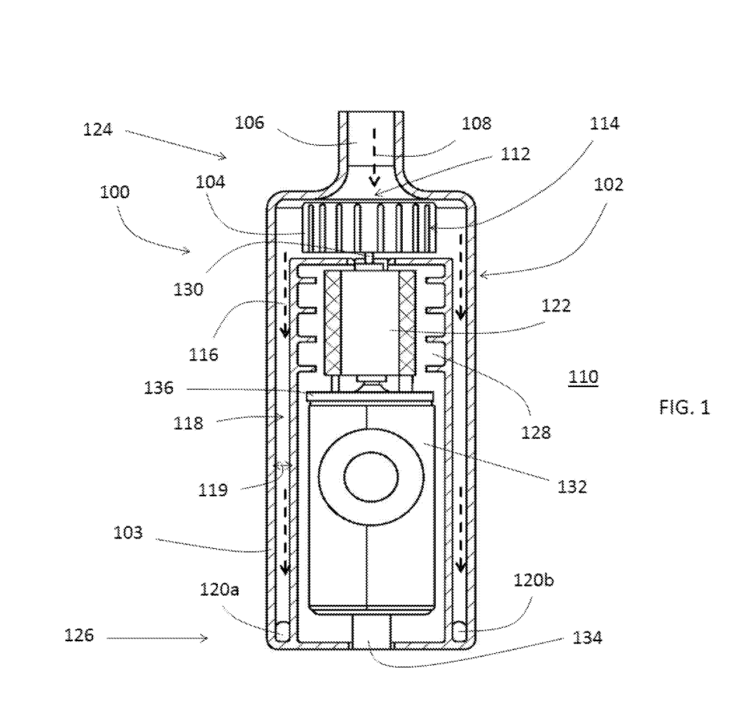

Referring now to FIG. 1, one embodiment of the present invention is shown in a cross-sectional side view. FIG. 1 shows several advantageous features of the present invention, but, as will be described below, the invention can be provided in several shapes, sizes, combinations of features and components, and varying numbers and functions of the components. The first example of an electronic portable aspirator 100, as shown in FIG. 1, includes a housing 102 and an impeller 104 disposed within the housing 102.

The housing 102 may be considered an aspirator housing that substantially surrounds the impeller 104. In the exemplary embodiment, the housing 102 is cylindrical in shape. In other embodiments, the housing 102 may be other shapes and sizes, such as, for example, L-shaped. The housing 102 is preferably formed as a waterproof, hermetically sealed housing so as to protect the electrical components housed therein from the outside environment. In a further embodiment, the electronic components may be coated with a hydrophobic coating. In another embodiment, the housing 102 may not be waterproof. In yet another embodiment, the aspirator 100 may be considered splash proof, meeting at least an IPX4 level of protection.

The housing 102 may include walls that are made of a plastic or other polymer-based material. In other embodiments, the housing 102 may be made of other materials. In one embodiment, the housing 102 may have an exterior wall 103 that is about 0.050″ to 0.100″ thick and may be 5.000″ to 7.000″ in length. In other embodiments, the housing 102 may have other dimensions. In a preferred embodiment, the housing 102 is sized and shaped to provide a portable hand-held aspirator that can be used on-the-go and stored and transported easily (e.g., being small and light enough to be conveniently storable within, for example, a diaper bag).

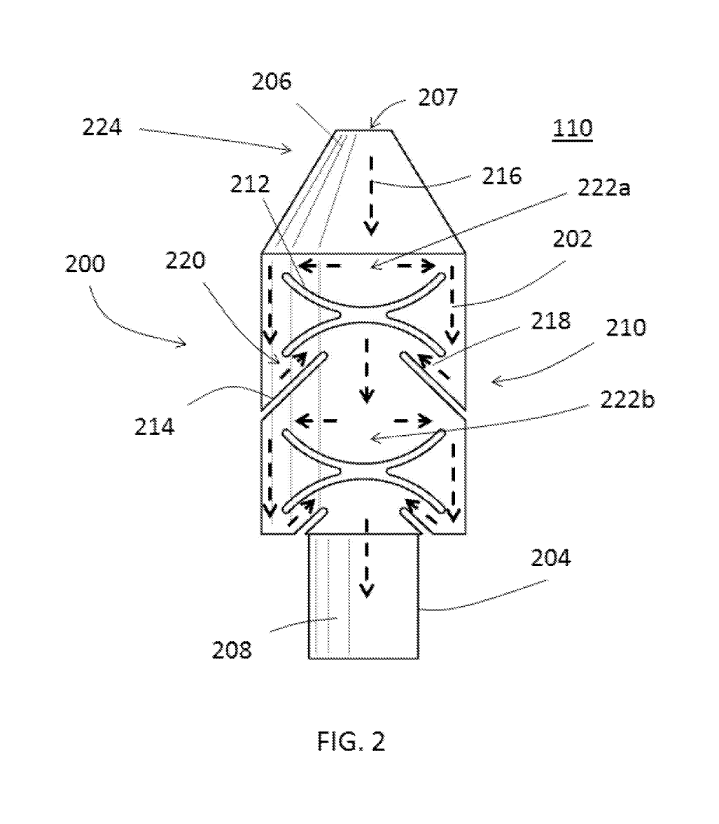

The housing 102 may include a suction inlet 106. The suction inlet 106 may be considered a relatively narrow passageway fluidically coupled to the impeller 104, through which a suction force 108 created by the impeller 104 draws-in/sucks-in air from the outside environment 110. In other words, the impeller 104 creates a negative pressure that draws-in air from the outside/ambient environment 110 through the suction inlet 106. Stated yet another way, the impeller 104 may have a suction side 106 opposite a discharge side through which air can be discharged back into the outside environment 110, as will be discussed in more detail herein below. The suction inlet 106 may be formed as a neck portion of the housing 102 and may be sized and shaped to couple to a bodily fluid removal element, such as, a disposable nasal tip 200 (FIG. 2). In one embodiment, the suction inlet 106 may have a 0.250″ diameter and may be about 1.000″ in length. In other embodiments, the suction inlet 106 may have other dimensions. In a preferred embodiment, the suction inlet 106 is configured to draw-in air only, and not bodily fluids (for easy clean-up and to prevent moisture exposure of the electrical components with the housing 102). As will be discussed with reference to FIG. 2, the bodily fluid removal element 200 is preferably configured to collect and retain bodily fluids within the bodily fluid removal element 200 such that bodily fluids do not enter the housing 102.

The impeller 104 preferably creates a suction force 108 that is greater than a typical force of a bulb syringe, diaphragm-pump aspirators, and other known aspirators. The impeller 104 is driven by a driving component, such as a DC motor 122, to create the suction 108 through the suction inlet 106. In one embodiment, the impeller 104 is fixedly coupled to a rotation shaft 130 of the motor 122. In one embodiment, rotation of the motor 122 may rotate the rotation shaft 130 and the impeller 104 to create the suction 108 through the suction inlet 106.

In one embodiment, the aspirator 100 operates at 12 volts, spinning the motor 122 at 30,000 revolutions-per-minute (RPMs), as measured at 0.500″ from its center. In other embodiments, the motor 122 may spin the impeller 104 at a rate of between 25,000-40,000 RPMs. Such high RPMs should create a suction force that is much stronger than a typical bulb syringe, diaphragm-pump aspirators, and other known aspirators. In yet other embodiments, the motor 122 may rotate the impeller 104 at RPMs outside of these ranges, but should generally rotate quickly enough to generate a strong suction force 108, as compared to known nasal aspirators, such as, for example, the bulb syringe and diaphragm-pump aspirators.

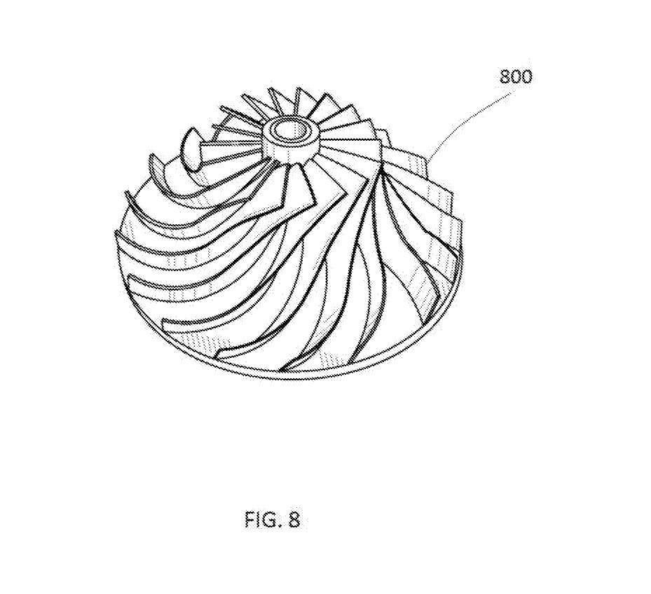

In one embodiment, the impeller 104 may be formed as a centrifugal compressor impeller 800, as shown in FIG. 8, for example. In other embodiments, the impeller 104 may be formed as another known type of impeller. In one embodiment, the impeller 104 may be made of a plastic resin or other polymer-based compound. The impeller 104 may be about 1.25″ in diameter and have a height that is within a range of 0.200″-0.500″. In other embodiments, the impeller 104 may have other dimensions, or be made of other known materials, but is preferably sized to provide a portable aspirator.

The impeller 104 may define an impeller inlet 112 disposed at a center of the impeller 104 (sometimes referred to in the art as an impeller's “eye”) and may also include a plurality of blades 114 disposed around the impeller inlet 112. The number of blades 114 may be any number. In one embodiment, the number of blades 114 is between 5-30 blades 114. In another embodiment, a thickness of the blades 114 may be within a range of 0.025″-0.050″. In other embodiments, the thickness of the blades 114 may be outside of this range.

The plurality of blades 114 may be operably configured to push air in a radially outward direction during a rotary motion of the impeller 104. Stated another way, the plurality of blades 114 may be operable to direct an airflow 116 (as indicated by the dotted lines shown in FIG. 1) created by the impeller 104 through an airflow discharge passage 118 and out to the outside environment 110, via at least one exhaust port 120. In the exemplary embodiment, the housing 102 defines two exhaust ports 120a-b. In other embodiments, the housing 102 may define more or less than two exhaust ports 120. The housing 102 may at least partially define the airflow discharge passage 118. Exterior housing walls and internal housing walls may define the airflow discharge passage 118, as shown in FIG. 1. In one embodiment, a width 119 of the airflow discharge passage 118 may be at least 0.025″ substantially along the length of the passage 118. The airflow discharge passage 118 fluidically couples the impeller 104 to the exhaust port 120 to discharge the airflow from the impeller 104 to the outside environment 110.

The rotary motion of the impeller 104 and the motor 122, while generating a stronger suction force 108 than many known aspirators, may also tend to create undesirable noise and vibration effects. Accordingly, in one embodiment, the position of the impeller 104 relative to the exhaust port 120 may be configured to reduce noise. For example, the impeller 104 may be disposed at a proximal end 124 of the housing 102 and the exhaust port 120 may be disposed at a distal end 126 of the housing 102, opposite the proximal end 124. The exhaust port(s) 120 should preferably be positioned a distance away from the impeller 104, without negatively affecting the suction 108. This is possible because air flows from high-pressure to low-pressure. To elaborate, the exhaust ports 120 should be areas of low-pressure, as compared to the high-pressure created by the impeller 104 sucking-in air at the proximal end 124 of the housing 102. Due to the position of the exhaust ports 120 a sufficient distance downstream of the impeller 104, the air flowing through the airflow discharge passage 118 should slow down by the time the air reaches the exhaust ports 120 downstream. This reduces the noise level of sound created by the high-speed movement of the impeller 104. Further, a thickness and/or material properties of the housing 102 walls may also be selected to further reduce the noise level for the patient.

In one preferred embodiment, the exhaust port 120 is disposed a sufficient distance from the impeller 104 and the motor 122 such that a noise from the impeller 104 and the motor 122 is below 85 decibels (as measured from a sound level meter that is less than 4 inches away from the aspirator 100). In a more preferred embodiment, the exhaust port 120 is disposed a sufficient distance from the impeller 104 and the motor 122 such that a noise from the impeller 104 and the motor 122 is within a range of 60-75 decibels. In yet other embodiments, the noise may be outside of this range, but should be generally below 85 decibels.

In another embodiment, vibration caused by the motor 122 may be reduced by the inclusion of vibration-dampening material 128 disposed around the motor 122. Vibration from the motor 122 may cause a tickling or other undesirable agitation of a patient's body part (e.g., nose). The vibration-dampening material 128 may be any known material operable to absorb vibrations, such as, for example, sorbothane, polyurethane foams, and other like materials. In other embodiment, the aspirator 100 may not include vibration-dampening material 128.

The portable electronic aspirator 100 further includes a power source 132. In one embodiment, the power source 132 is a rechargeable battery (e.g., Li-Ion 14500 cell) disposed within the housing 102. The power source 132 is electrically coupled to the motor 122 to drive rotation of the impeller 104. The power source 132 may be rechargeable via, for example, a power connector 134 (e.g., 2.1 mm jack, USB connector, etc.) disposed to couple to, for example, a wall charger. In one embodiment, the rechargeable batteries 132 may output 12 Vac, when fully charged, to provide the motor 122 with the power to reach the RPMs required for optimal suction. In a further embodiment, the aspirator 100 may further include a protection circuit 136 operably configured to prevent over-charging the power source 132 and prevent electrical damage due to current spikes. In one embodiment, the protection circuit 136 may be configured with a shut-off voltage that prevents the power source 132 from dropping below the optimal suction voltage. In one embodiment, the protection circuit 136 is configured with a shut-off voltage of, for example, 9 Vac. In certain embodiments, RPMs of the motor 122 significantly drop when power drops below 9 Vac (resulting in a less than optimal suction force at the suction inlet 106). In yet a further embodiment, one or more resistors may be included in the circuit between the power source 132 and the motor 122 to limit an in-rush current.

Referring now primarily to FIG. 2, with reference also to FIG. 1, the disposable tip 200 will now be described. Advantageously, the disposable tip 200 may be configured to collect and retain viscous bodily fluids (e.g., mucous) sucked-in to the aspirator 100 by the impeller 104 so that such bodily fluids do not enter the housing 102. This provides a benefit over the existing art of easy clean-up, as well as, isolation and protection of the electrical components from aspirated bodily fluids. Inventively, the disposable tip 200 may collect and retain aspirated bodily fluids (without requiring the use of known replacement filters) by changing a direction of a flow of the aspirated bodily fluids 202, as will be described in more detail herein below.

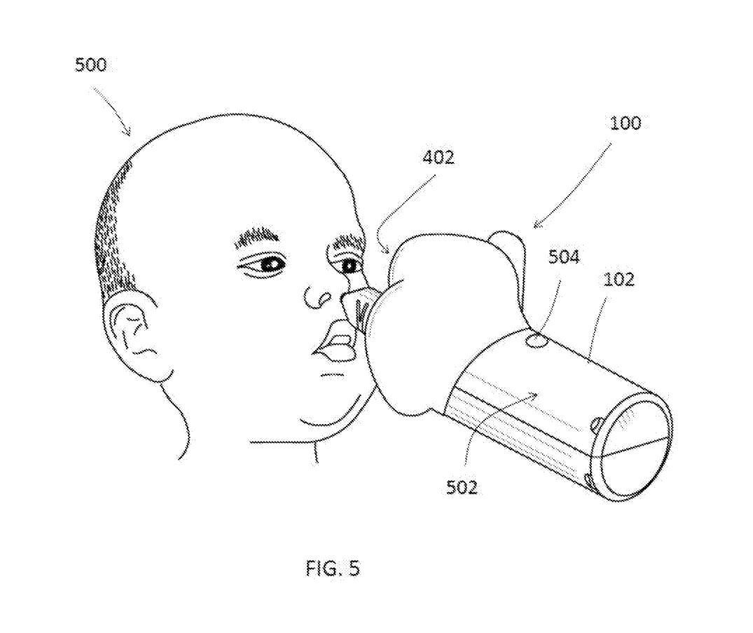

As used herein, the term “disposable” is intended broadly to indicate an article or device that is configured to be used once, or a few times, or until no longer useful, and then thrown away. In one embodiment, the disposable tip 200 may be more narrowly configured for single-use application. In yet further embodiments, the disposable tip 200 may be made of a biodegradable material, such as, for example, a PLA or HDPE with biodegradable additives. The term “tip,” as used herein, is intended to indicate a pointed, rounded, tapered, or otherwise narrow (as compared to the housing 102) end or attachment that is configured and dimensioned for insertion, of at least a portion thereof, within a human body orifice (e.g., nose). FIG. 5 illustrates one example of this feature, showing an absolute end of the disposable tip 200 inserted within a child's nasal opening.

The disposable tip 200 may be dimensioned and configured to couple to the housing 102 at the suction inlet 106. In one embodiment, the disposable tip 200 may include a coupling element 204. The coupling element 204 may be dimensioned and configured to matingly engage the suction inlet 106 by, for example, a press fit coupling. For example, a diameter of the coupling element 204 may be sized to be the same, or slightly larger than a diameter of the suction inlet 106 to couple the housing 102 to the tip 200 by a friction fit. In other embodiments, the disposable tip 200 may be removeably couplable to the housing 102 in other known ways and using other known fastening devices. The disposable tip 200 may, in an alternative embodiment, be fixedly coupled to the housing 102 at the suction inlet 106.

The disposable tip 200 defines a fluid intake port 206 that, when coupled to the housing 102, is configured to be in fluid communication with both the outside environment 110 and the suction inlet 106. More specifically, the suction force 106 from the impeller 104 draws-in air from the outside environment 110 through the fluid intake port 206 of the tip 200 and through the suction inlet 106 of the housing 102. The design of the disposable tip 200 should include fluid passageways that permit the free flow of air from the outside environment 110 (i.e., not materially inhibiting the suction force) to the impeller 104; yet be simultaneously configured to trap and retain viscous bodily fluids within the tip 200.

In one embodiment, the disposable tip 200 may define a single fluid intake port 206. In other embodiments, the disposable tip 200 may define more than one fluid intake port 206. For example, in some embodiments, the disposable tip 200 may define a distal central intake port at a nasal insertion portion of the tip 200 to draw-in mucous, as well as, a plurality of side intake ports disposed to draw-in additional ambient air from the outside environment 200.

In one embodiment, each fluid intake port 206 may define at least one entrance aperture 207. The entrance aperture 207 may have a diameter that is about 0.150″. In other embodiments, the entrance aperture 207 may be larger or smaller than 0.150″ in diameter. The disposable tip 200 may also include a fluid exit port 208 that may be disposed opposite to and/or concentric with the fluid intake port 206. Between the fluid intake port 206 and the fluid exit port 208, an aspirated bodily fluid collection region 210 may be disposed to collect and retain aspirated bodily fluids within the tip 200, while simultaneously permitting air from the outside environment 110 to flow through to the impeller 104 within the housing 102. Preferably, the design of the disposable tip 200 should trap viscous bodily fluids therein, without substantially obstructing the suction air flow.

In one embodiment, the disposable tip 200 retains the aspirated bodily fluids by changing the direction of the flow of the aspirated bodily fluids 202. As used herein, the phrase “changing the direction of the flow of the aspirated bodily fluids” is intended to indicate that the flow continues to move, but in a different direction. Stated another way, the phrase “changing the direction of the flow of the aspirated bodily fluids” is not intended to encompass sponge-type filters that merely stop the movement of mucous through a tube.

In one embodiment, the aspirated bodily fluids collection region 210 may include at least one barrier 212 that changes or redirects the flow of aspirated bodily fluids 202. In a further embodiment, the barrier 212 may be formed as a U-shaped wall, with the “U” curving towards the fluid exit port 208, as shown in FIG. 2. Stated another way, the barrier 212 may be a concave-shaped wall, opening toward the fluid intake port 206. In yet a further embodiment, the barrier 212 may be formed as one or more “X”-shaped walls disposed in a center of the collection region 210 to split the fluid flow into at least two separate, divergent paths, as shown in FIG. 2. In one embodiment, each of the barriers 212 may be about 0.300″ in length, with the diameter of the collection region 210 being about 0.500″. In other embodiments, the dimensions of the barriers 212 and the collection region 210 may be outside of this range.

In yet a further embodiment, a second set of barriers 214 may be disposed downstream of the barrier 212 and may be disposed to reverse a direction of the flow of fluids 202. In other words, the barrier(s) 214 may reverse the direction of the flow of aspirated bodily fluids 202 such that the flow becomes directed towards the entrance aperture 207 (or fluid intake port 206), rather than away from it. This concept of reversing the direction of fluid flow may be illustrated by a comparison of the arrows 216 (illustrating an initial direction away from the entrance aperture 207) and 218 (illustrating a reversal direction towards the entrance aperture 207). By reversing the direction of the flow of aspirated bodily fluids 202 within the collection region 210, such fluids are retained within the disposable tip 200, rather than entering the housing 102. In one embodiment, the barrier(s) 214 may be considered to be walls that are angled toward the entrance aperture 207. Such barrier walls 214 may, for example, be angled between 5 degrees to 85 degrees generally toward the entrance aperture 207.

The barrier walls 214 may be substantially flat, smooth, and/or planar. In other embodiments, the barrier walls 214 or 212 may be slightly curved, or rectilinear in shape. In other embodiments, such shapes and dimensions may vary, but should generally be configured to change the direction of flow of viscous bodily fluids within the disposable tip 200 thereby retaining or trapping such bodily fluids therein.

In one embodiment, the aspirated bodily fluids collection region 210 may be dimensioned and configured to direct the flow of aspirated bodily fluids 202 in a zig-zag pattern within the disposable tip 200, as shown in the exemplary embodiment depicted in FIG. 2. As used herein, the term “zig-zag” is intended to indicate a path of fluid movement having abrupt alternative generally left and right turns. In another embodiment, the aspirated bodily fluids collection region 210 may be considered a multi-directional chamber that defines a multidirectional bodily fluid passageway 220 (as indicated by the arrows in FIG. 2). The multidirectional bodily fluid passageway 220 may be a continuous passageway with multiple, separate portions along its length. Each such portion may direct the fluid flow in a different direction than the immediately previous portion. In one embodiment, the multidirectional bodily fluid passageway 220 may have at least two separate portions along its length that at least partially overlaps within one another within the disposable tip 200. For example, a first portion 222a overlaps with a second portion 222b within the disposable tip 200. Such embodiments of the collection region 210 discussed herein inventively retain the bodily fluids within the disposable tip 200, without requiring a replacement filter, by changing a direction of the fluid flow 202. Accordingly, the wall barriers 212 and 214 may also be considered non-filter barriers 212, 214.

In a preferred embodiment, the disposable tip 200 is dimensioned and configured for insertion with a human nostril, preferably being small enough for insertion with a human infant's nostril. Thus, in preferred embodiments, at least a portion of the disposable tip 200 may be formed as a nasal insertion tip portion. For example, an absolute end 224 (opposite the coupling element 204) of the disposable tip 200 may have a diameter or width that is smaller than a diameter or width of an average-sized human infant's nostril. It is contemplated that in alternative embodiments, the disposable tip 200 may be configured and dimensioned for insertion within other human orifices, within which it may be desirable to remove other bodily fluids.

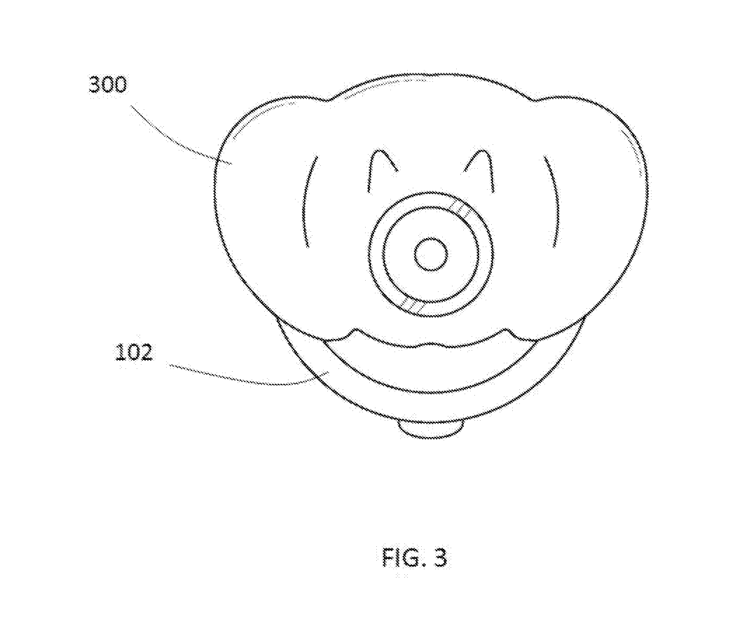

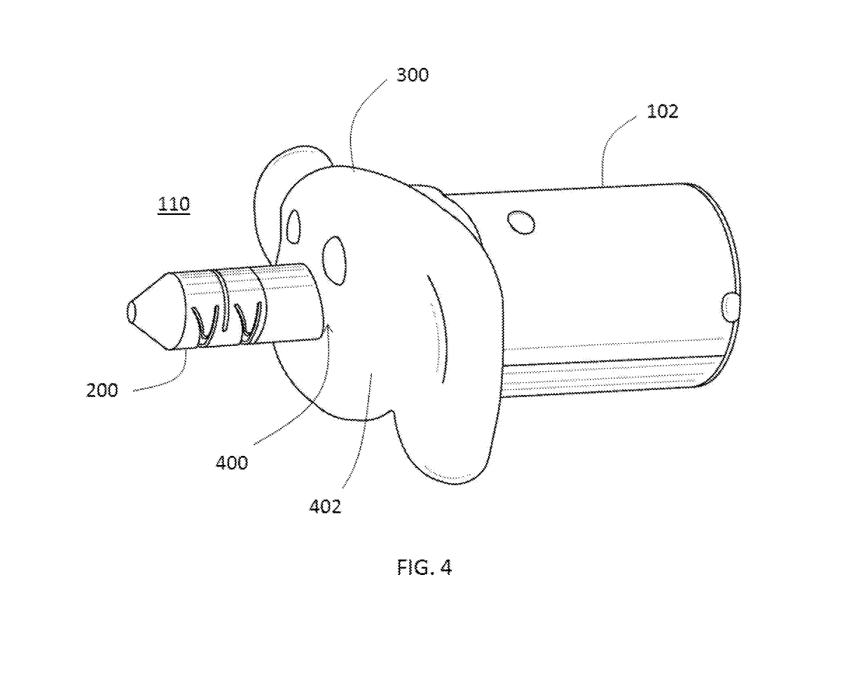

Referring now primarily to FIGS. 3-5, with brief reference to FIGS. 1 and 2, other features of embodiments of the aspirator 100 are described. In one embodiment, the aspirator 100 may include a decorative body 300. The decorative body 300 may be removeably couplable to the housing 102 at, for example, the suction inlet 106. The decorative body 300 may define an opening 400 sized to slideably receive at least a portion of the disposable tip 200 through the opening 400 into the outside environment 110. The decorative body 300 may provide a welcome distraction for a patient 500 (e.g., child). Accordingly, in one embodiment, the decorative body 300 may include an animal face surface 402 that, when coupled to the housing 102, is disposed to face in a direction away from the housing 102 and toward the patient 500. In other words, the animal face surface 402, when coupled to the housing 102, should be viewable by the patient 500 during a nasal aspiration use-configuration, as shown in FIG. 5. The word “animal,” as used in the phrase “animal face surface,” is intended broadly to encompass human characters as well as non-human faces (e.g., non-human and human character faces). Advantageously, the patient 500 may be emotionally comforted, or at least momentarily distracted, by the view of the animal face surface 402 as bodily fluids are being extracted. In another embodiment, the surface 402 may be a generally decorative surface (e.g., flowers, or multi-colored patterns), rather than an animal face surface 402.

In one embodiment, the portable electronic aspirator 100 may be provided in the form of an aspirator kit. The aspirator kit may include the housing 102 surrounding the impeller 104, the motor 122, and the other electronic components and fluid passageways, as described herein above with reference to FIG. 1. The aspirator kit may further include at least one disposable tip 200. In a further embodiment, the aspirator kit may further include two or more disposable single-use tips 200. In yet a further embodiment, the aspirator kit may further include one or more interchangeable decorative bodies 300a-n. For example, a first decorative body 300a within the aspirator kit may have an animal face surface 402 resembling a first cartoon character and a second decorative body 300b within the aspirator kit may have an animal face surface 402 resembling a second cartoon character, different from the first. Accordingly, a child-friendly aspirator kit may be provided with a very strong suction force and interchangeable decorative bodies 300a-n. Further, the aspirator kit may be operator-friendly in that after a single-use, the disposable single-use tip 200 may be thrown away, eliminating the need for clean-up.

The housing 102 may also be dimensioned and configured as an operator handle portion. Stated another way, an exterior surface 502 of the housing 102 may be formed as a user gripping surface disposed to receive an operator's hand grip. As shown in FIG. 5, the housing 102 may double as a handle for a parent to hold the aspirator 100 up to a child's nose during extraction. Children may squirm during use. Accordingly, it would be beneficial to shape the housing 102 so as to provide a handle portion for the parent to grip the aspirator 100 with one hand (easily guiding and holding the tip 200 in the nostril) while holding the child still with the other hand.

The aspirator 100 may also include an actuator button 504 disposed on the housing 102. The actuator button 504 may be coupled to the electronic components within the housing 102 and may be operable to selectively rotate the motor 122 and the impeller 104 to create the suction force 108 at the suction inlet 106. The actuator button 504 may be preferably disposed on the handle portion of the housing 102 at a position that provides for convenient pressing of the button 504 while the operator is gripping the handle portion of the housing 102.

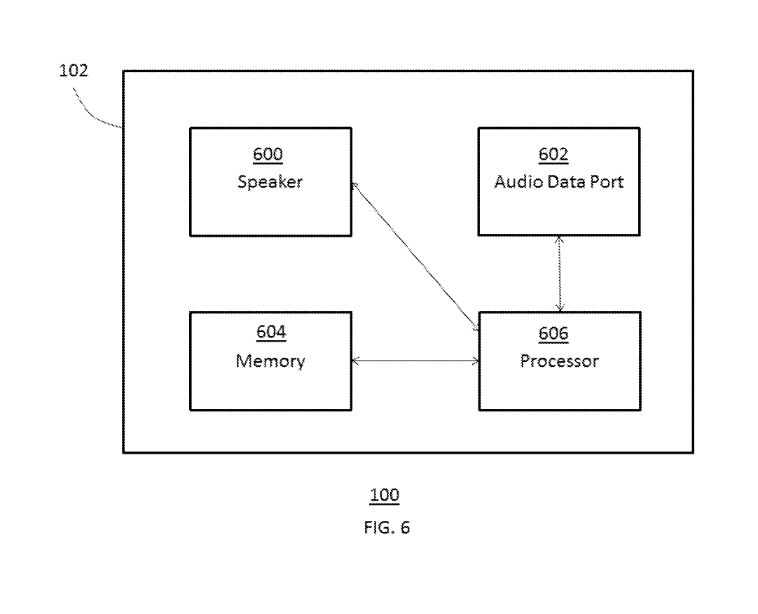

Referring now primarily to FIG. 6, with brief reference to FIGS. 1 and 5, yet another embodiment of the portable electronic aspirator 100 is illustrated in a block diagram view. FIG. 6 depicts a portion of electronic components that may be disposed within the housing 102 in one embodiment of the present invention. In such embodiment, the aspirator 100 may include a speaker 600, an audio data port 602, a memory 604, and a processor 606. The processor 606 may be operably configured to receive one or more music files via the audio data port 602 and cause the music files to be played, i.e., emitting audio signals corresponding to the music files through the speaker 600. The memory 604 may store the music files in a non-transitory memory. The memory 604 may be separate from the processor 606, or may be memory within the processor 606. In one embodiment, there may also be a user input interface that allows the user to select one from among a plurality of music files that may be played for the patient 500. Accordingly, the patient 500 may further be distracted and/or comforted by the music being played through the speakers 600.

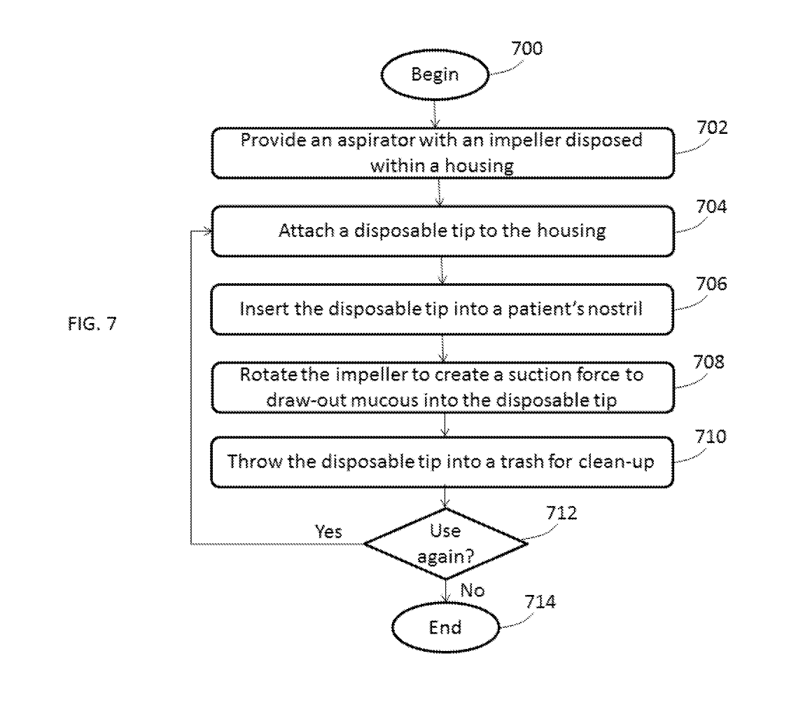

The process flow chart of FIG. 7, illustrating exemplary use of the aspirator 100, will now be described with reference also to FIGS. 1-6. Although FIG. 7 shows a specific order of executing the process steps, the order of executing the steps may be changed relative to the order shown in certain embodiments. Also, two or more blocks shown in succession may be executed concurrently or with partial concurrence in some embodiments. Certain steps may also be omitted in FIG. 7 for the sake of brevity.

The process may begin in step 700 and may immediately proceed to step 702, where the operator (e.g., a parent or a medical professional) may be provided with the impeller 104 disposed with the housing 102. In step 704, the operator may attach the disposable tip 200 to the housing 102. In step 706, the operator may insert at least a portion of the disposable tip 200 within the patient's 500 nostril. In step 708, the operator may turn-on the impeller 104, thereby rotating the impeller 104 to create the suction force 108 sufficient to draw-out mucous from the nasal cavity into the disposable tip 200. In step 710, after the mucous has been drawn-out of the nasal cavity, the operator may turn-off the impeller 104 and remove and discard the disposable tip 200. In step 712, the operator may decide whether to use the aspirator 100 at a subsequent instance/time. If the answer is “yes,” the process may proceed back to step 704, where the operator may attach another (unused) disposable tip 200. The process may repeat steps 704 through 712 again. If the answer is “no,” the process may proceed to step 714, where the process may immediately end.

A novel and efficient electronic aspirator has been disclosed that increases the suction strength over existing aspirators, while also providing for easy and efficient clean-up. Embodiments of the invention provide an electronic portable aspirator that includes an impeller, which provides a suction force that is stronger than conventional bulb syringes, diaphragm-pump aspirators, and other known aspirators. In addition, embodiments of the invention provide an aspirator with a disposable tip removeably couplable to the housing of the aspirator and designed to trap bodily fluids, such as mucous, within the disposable tip for easy clean-up and so that such bodily fluids do not enter the aspirator housing. Additional embodiments of the invention provide for a removable and interchangeable decorative face that can comfort patients (e.g., children) and/or an audio system that can selectively upload and play music files to further comfort the patient during the bodily fluid removal process. Yet other embodiments of the invention provide for noise reduction and vibration dampening features.

What is claimed is:

1. An aspirator comprising:
a housing having a suction inlet;
an impeller disposed within the housing, the impeller being driven by a driving component to create a suction through the suction inlet; and
a disposable tip that is removeably couplable to the housing at the suction inlet and includes a fluid intake port at a distal end of the disposable tip and a fluid exit port at a proximal end at the suction inlet and having a passage between the intake port and fluid exit port, the disposable tip further including an aspirated bodily fluid collection region within the passage including at least one barrier within the passage that changes direction of a flow of the aspirated fluids within the fluid collection region and retains the aspirated fluids within the fluid collection region.

2. The aspirator in accordance with claim 1, wherein:
at least a portion of the disposable tip is formed as a nasal insertion tip, the nasal insertion tip being dimensioned and configured for insertion with a nostril.

3. The aspirator in accordance with claim 1, wherein:
the aspirated bodily fluid collection region includes multiple barriers disposed to direct a flow of the aspirated bodily fluids to reverse direction within the disposable tip.

4. The aspirator in accordance with claim 1, wherein:
the aspirated bodily fluid collection region includes X-shaped barriers disposed to direct a flow of the aspirated bodily fluids in a zig-zag pattern within the disposable tip.

5. The aspirator in accordance with claim 1, wherein:
the aspirated bodily fluid collection region includes a multidirectional chamber defining a multidirectional bodily fluid passageway within the disposable tip, the multidirectional bodily fluid passageway having at least two separate portions along a length of the passageway that at least partially overlap one another within the disposable tip.

6. The aspirator in accordance with claim 1, wherein the impeller:
includes a plurality of blades and is fixedly coupled to a rotation shaft of the driving component.

7. The aspirator in accordance with claim 1, wherein:
the impeller defines a center inlet and includes a plurality of blades disposed around the center inlet, the plurality of blades being operably configured to push air in a radially outward direction during a rotary motion of the impeller.

8. The aspirator in accordance with claim 1, wherein:
the housing at least partially defines an airflow discharge passage fluidly coupling the impeller to at least one exhaust port disposed at a distal end of the housing opposite a proximal end of the housing at which the impeller is disposed; and
the impeller includes a plurality of blades operable to direct airflow created by the impeller through the airflow discharge passage and to the outside environment via the at least one exhaust port.

9. The aspirator in accordance with claim 1, wherein:
the driving component is formed as a motor; and
the housing includes at least one exhaust port at a distal end of the housing, the at least one exhaust port disposed a sufficient distance from the impeller and the motor such that noise from the impeller and the motor is below 85 decibels.

10. The aspirator in accordance with claim 1, further comprising:
a vibration-dampening material disposed around the driving component and configured to absorb vibrations created by the driving component.

11. The aspirator in accordance with claim 1, further comprising:
a decorative body removeably couplable to the housing at the suction inlet and defining an opening for receiving at least a portion of the disposable tip through the opening into the outside environment.

12. The aspirator in accordance with claim 11, wherein:
the decorative body includes an animal face surface disposed to face away from the housing such that the animal face surface is viewable by a patient in a nasal aspiration use-configuration.

13. The aspirator in accordance with claim 1, further comprising:
a speaker, an audio data port, and a processing device at least partially disposed within the housing, the processing device operably configured to receive a music file via the audio data port and cause audio signals associated with the music file to emit through the speaker.

14. The aspirator in accordance with claim 1, wherein:
the housing includes an exterior surface, at least a portion of the exterior surface formed as a handle portion with a user gripping surface disposed to receive an operator's hand grip to guide and hold at least a portion of the disposal tip into a patient's nostril.

15. The aspirator in accordance with claim 1, further comprising:
an actuator button disposed on the housing and operable to selectively rotate the impeller to create the suction.

16. A disposable nasal tip for an aspirator, comprising:
a coupling element configured to removeably couple the disposable nasal tip to the aspirator at a suction inlet of the aspirator;
a fluid intake port in fluid communication with an outside environment; and
an aspirated bodily fluid collection region, formed in the disposable nasal tip between the fluid intake port and a fluid exit port, including non-filter barriers configured to collect and retain aspirated bodily fluids drawn into the disposable nasal tip by a suction force from the aspirator, the non-filter barriers retaining the aspirated bodily fluids by changing a direction of a flow of the aspirated bodily fluids within the disposable nasal tip.

17. The disposable nasal tip in accordance with claim 16, wherein:
the non-filter barriers are disposed to direct a flow of the aspirated bodily fluids to reverse direction within the disposable nasal tip.

18. The disposable nasal tip in accordance with claim 16, wherein:
the non-filter barriers are disposed to direct a flow of the aspirated bodily fluids in a zig-zag pattern within the disposable nasal tip.

19. An aspirator kit comprising:
an aspirator device including a suction inlet, an impeller driven by a driving component to create a suction through the suction inlet, and a housing surrounding the impeller and the driving component; and
at least one disposable tip, each of the at least one disposable tip that is removeably couplable to the housing at the suction inlet and defines a fluid intake port in fluid communication with an outside environment and the suction inlet, and that has an aspirated bodily fluid collection region that collects and retains aspirated bodily fluids drawn into the disposable tip by the impeller's suction by changing a direction of a flow of the aspirated bodily fluids within the disposable tip.

20. The aspirator kit in accordance with claim 19, further comprising:
at least one decorative body, each of the at least one decorative body being removeably couplable to the aspirator device and defining an opening for receiving at least a portion of the disposable tip through the opening into the outside environment; and
wherein:
each of the at least one decorative body includes a decorative surface facing away from the housing and each of the least one decorative body having a different decorative surface from the other ones of the at least one decorative body.
